(19) United States
(12) Patent Application Publication
(10) Pub. No.: US 2006/0036133 A1
(43) Pub. Date: Feb. 16, 2006

(54) Oral examination tool

(76) Inventor: Christopher Demsky, Watkinsville, GA (US)

Correspondence Address: Donald W. Meeker;Patent Agent, 924 East Ocean Front #E, Newport Beach, CA, 92661, US

(21) Appl. No.: 10/919,177

(22) Filed: Aug. 16, 2004

(51) Int. Cl. A61B 1/32

(52) U.S. Cl. 600/240

(57) ABSTRACT

A hand-held plastic oral examination tool has a spoon-shaped head on a first end with a rim for resting on a human tongue outside the central gag reflex sensitive area to prevent a gag reflex. A second end has one textured non-slip surface for contacting slippery surfaces and an opposite smooth surface for contacting sensitive areas.

### BACKGROUND OF THE INVENTION

1. Field of the Invention

The present invention relates to oral examination tools and particularly to a plastic oral examination tool with a spoon-shaped first end and a second end having one textured non-slip surface and an opposite smooth surface.

2. Description of the Prior Art

Oral examinations require a tool to move tongue or lips or cheeks out of the line of vision during the oral examination. Each of the different surfaces to be contacted has different textures and sensitivities. It is important that the oral examination tool does not slip during use or irritate sensitive tissue or create a gag reflex.

Tongue depressors are normally employed to hold the tongue or lips or cheeks out of the line of vision during an oral examination. The ordinary tongue depressor is a flat wooden stick with rounded ends. The wood offers a non-slip surface, but the taste is usually unpleasant and often the dryness and texture of the wood and the flat surface cause a gag reflex from the patient. The majority of the nerves that create the gag reflex are located in the center of the tongue.

Furthermore, manufacturing the wooden tongue depressors requires cutting, shaping, and sterilizing the wooden sticks under pressure.

Prior art devices have not adequately addressed the need for a tool, which can be used in the mouth to hold various surfaces without slipping and without creating a gag reflex or irritating the surfaces, contacted.

U.S. Pat. No. 2,653,597, issued Sep. 29, 1953 to Canan, puts forth a tongue depressor, which comprises a flat strip of plastic having one end that is spoon-shaped and wider than the shaft. The other end of the tongue depressor is provided with a small rectangular mirror.

U.S. Pat. No. 5,553,627, issued Sep. 10, 1996 to Newkirk, puts forth an improved tongue depressor that has a flat elongated blade having a first end and a second end. To the first end is integrally attached a first tongue-contact section and to the second end is integrally attached a second tongue-contact section. Each of the tongue-contact sections has a lower surface that includes sequentially, a downward slope, a flat horizontal surface, and an upper slope. The downward slope and the flat horizontal surface interface with the tongue and preferably have a non-slip surface. At the center and lower horizontal surface of the elongated blade is located a reinforcing protrusion that prevents the tongue depressor from bending or breaking in the middle. Because the size of an adult mouth is generally larger than a child's mouth, one of the tongue-contact sections is made with a smaller width. Also, the tongue depressor is constructed of a plastic. Therefore, the aftertaste experienced by patients when using a wooden tongue depressor is eliminated.

U.S. Pat. No. D263,743, issued Apr. 6, 1982 to Priestman, is for the ornamental design for a tongue depressor, which has a textured handle.

U.S. Pat. No. D337,158, issued Jul. 6, 1993 to Summers, concerns the ornamental design for an offset tongue depressor, which has a textured surface on each end.

U.S. Pat. No. D344,335, issued Feb. 15, 1994 to Elisha, illustrates the ornamental design for an angularly bent tongue depressor, which is bowed widthwise and has a textured surface at each end.

U.S. Pat. No. D217,410, issued Apr. 28, 1970 to Greatman, discloses the ornamental design for a tongue depressor, which has indentations on the top and bottom of both ends of the device.

U.S. Pat. No. 1,396,933, issued Nov. 15, 1921 to Jacoby, discloses a disposable tongue depressor created from vulcanized or hard fiber, preferably paper. The tongue depressor is arched laterally, having a strengthening rib, to resist bending along the length thereof.

U.S. Pat. No. D52,766, issued Dec. 17, 1918 to Jacoby, describes the ornamental design for a tongue depressor, which is laterally curved.

U.S. Pat. No. D359,556, issued Jun. 20, 1995 to Hale, indicates the ornamental design for an oral examination tongue depressor, which has an arcuate body and textured surfaces on the bottom of each end.

U.S. Pat. No. 5,518,503, issued May 21, 1996 to Rooney, shows an oral examination tongue depressor. The elongated relatively thin longitudinally accurately bowed disposable tongue depressor has the major portion of its length characterized by a transverse wide substantially V-shape and having a flesh adhesive texture on the respective end portions of its ventral surface.

U.S. Pat. No. 3,768,477, issued Oct. 30, 1973 to Anders, concerns a spoon-shaped tongue depressing aspirating tip.

U.S. Pat. No. 5,656,014, issued Aug. 12, 1997 to Rooney, provides an oral examination illuminating tongue depressor. The illuminated tongue depressor is formed by an elongated relatively narrow body having a handle end portion containing a battery and a proximal end portion having a curvature conforming to the larynx defining a convex top surface and a concave ventral surface having a friction inducing anti-slip texture adjacent its proximal end. A lamp imbedded in the top surface is connected with the battery by wiring through a switch. A sanitary disposable sheath envelopes the proximal end portion of the body.

What is needed is an oral examination tool for use in oral examinations which does not induce a gag reflex and which provides different surfaces for contacting different surfaces within the mouth.

### SUMMARY OF THE INVENTION

An object of the present invention is to provide an oral examination tool with a variety of differently textured and differently shaped surfaces adapted for contacting different surfaces within the mouth without causing irritation or slipping and without creating a gag reflex.

A related object of the present invention is to provide a plastic oral examination tool with a spoon-shaped head at a first end having a rim to prevent a gag reflex by contacting an area of the tongue outside of the center gag reflex area of the tongue by using only the rim of the spoon-shaped head and a second end having one textured non-slip surface for contacting slippery surfaces and an opposite smooth surface for contacting sensitive areas.

One more object of the present invention is to provide a plastic oral examination tool, which is easy and inexpensive to manufacture by hot injection molding requiring no sterilization.

An additional object of the present invention is to provide an oral examination tool with multiple surfaces for an easy to use single tool for multiple surfaces of the mouth.

A further object of the present invention is to provide an oral examination tool that is plastic and non-absorbent when placed in the mouth.

A contributory object of the present invention is to provide an oral examination device that can comfortably fit in the palm of a doctor or dentist's hand while maintaining a steady grip.

An added object of the present invention is to produce an oral examination device that doesn't leave an unpleasant taste in one's mouth.

In brief, a hand held oral examination tool has a spoon-shaped head on a first end having a rim to prevent a gag reflex by contacting an area of the tongue outside of the center gag reflex area and second end having one textured non-slip surface for contacting slippery surfaces of the mouth and an opposite smooth surface for contacting sensitive areas of the mouth.

An advantage of the present invention is to provide an oral examination tool without causing irritation or slipping and without creating a gag reflex.

Another advantage of the present invention is to provide an oral examination tool for contacting slippery surfaces and sensitive areas.

An additional advantage of the present invention is to provide a plastic oral examination tool, which is easy and inexpensive to manufacture requiring no sterilization.

One more advantage of the present invention is to provide an oral examination tool for an easy to use single tool for multiple surfaces of the mouth.

Yet another advantage of the present invention is to provide an oral examination tool that is not moisture absorbent.

Still another advantage of the present invention is to provide an oral examination device that can easily fit into the palm of a doctor or dentist's hand while maintaining a steady grip.

A further advantage of the present invention is to provide an oral examination device that does not produce an unpleasant taste in one's mouth.

### BRIEF DESCRIPTION OF THE DRAWINGS

These and other details of my invention will be described in connection with the accompanying drawings, which are furnished only by way of illustration and not in limitation of the invention, and in which drawings:

FIG. 1 is a perspective view of the oral examination tool of the present invention showing an upper side with a protruding hemisphere patterned textured end surface on one end and a textured interior spoon surface on the other end;

FIG. 2 is a top plan view of the invention of FIG. 1 showing the top surface;

FIG. 3 is a side elevational view of the oral examination tool of FIG. 1 showing the spoon end and the opposite end with a smooth surface and a textured surface;

FIG. 4 is a top plan view of the oral examination tool of the present invention showing the top surface with a smooth spoon interior on one end and a protruding pyramid patterned texture on an upper surface of an opposite end;

FIG. 5 is a side elevational view of the oral examination tool of FIG. 4 showing the spoon end and the opposite end with a smooth surface and a textured surface;

FIG. 6 is a bottom plan view of the invention of FIG. 4 showing the smooth outside of the spoon and a smooth surface at the opposite end.

### BEST MODE FOR CARRYING OUT THE INVENTION

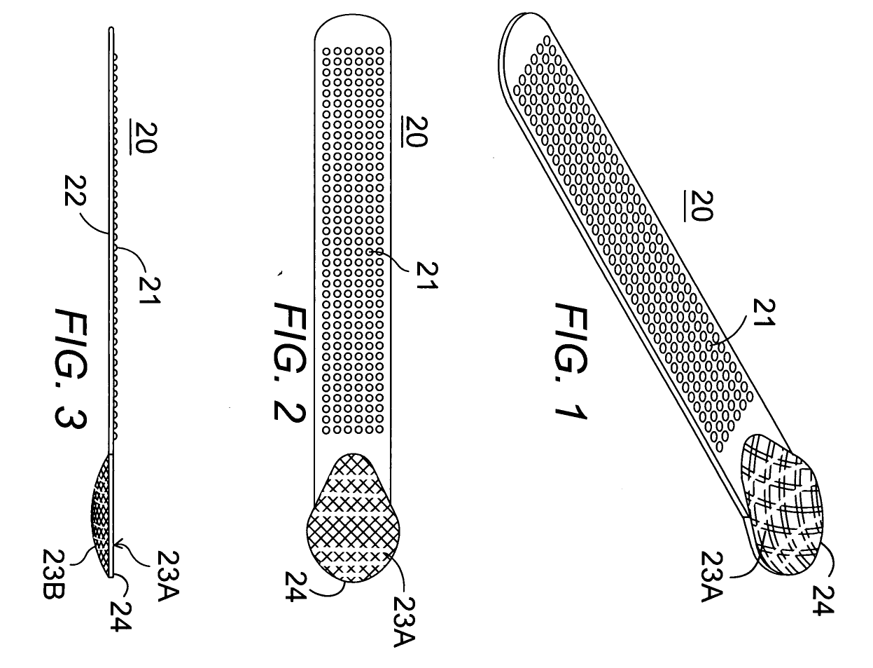

In FIGS. 1-6, an oral examination tool has a variety of differently textured and differently shaped surfaces for contacting a variety of surfaces within a human mouth.

An elongated substantially flat tool body 20 has a first end with straight parallel sides, a width narrower than a human tongue, and a rounded end. The first end is adapted for fitting with in a human mouth and consists of a smooth low friction surface 22 on one side of the tool body that is adapted for contact with sensitive surface areas with in a human mouth and a textured high friction surface 21 on the opposite side of the tool body adapted for contact with slippery areas on the mouth (as in FIG. 3). The tool body has a second end structured in the shape of a spoon head 23A and 23B that is wider than the first end and may have a textured or smooth spoon head as desired. The second end has a top concave side of the spoon head 23A having a rim 24 formed in a curve with the curved sides extending outwardly beyond the straight parallel sides of the first end of the tool body 20. The spoon rim 24 has a curved end that surrounds a recessed spoon interior surface 23A on one side of the second end of the tool body (as in FIGS. 1, 2, and 4). The spoon rim 24 is adapted for depressing a human tongue with out contacting a center gag reaction part of the tongue. The second end has a convex spoon exterior 23B on the opposite side of the second end. The spoon exterior 23 B is adapted for fitting within a palm of a user holding the tool body 20. The tool body adapted for being formed of a synthetic material, such as plastic, in a single piece by a hot molding process requiring no sterilization.

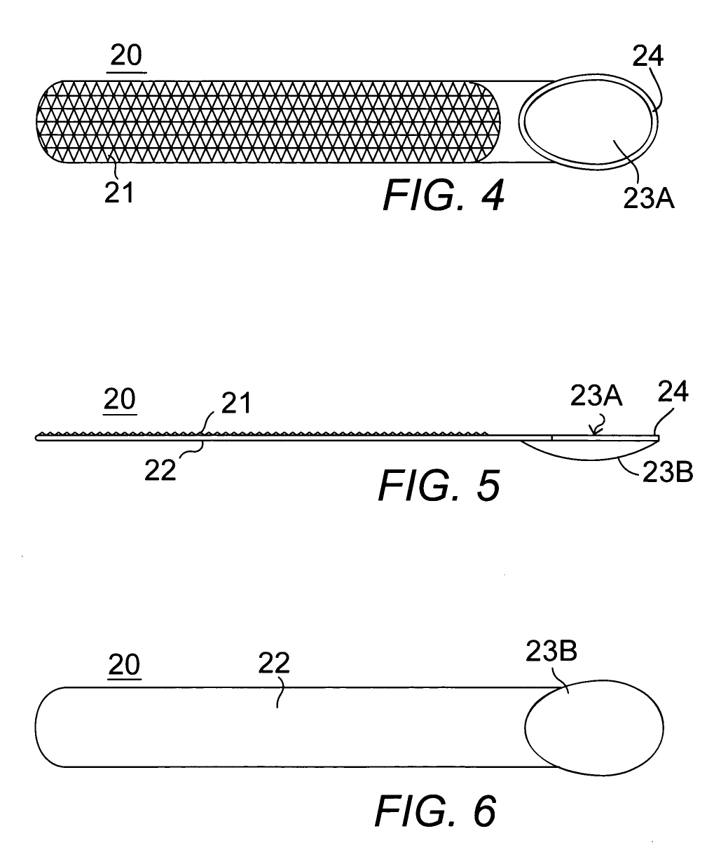

In FIGS. 1-3, the textured surface 21 comprises a pattern of protruding hemispheres and in FIGS. 4 and 5, the textured surface 21 comprises a pattern of protruding pyramids. In FIGS. 1 and 2, the interior surface of the spoon 23A is textured and in FIG. 3, the spoon exterior 23B is also textured. In FIG. 4 the spoon interior 23A surface is smooth and in FIGS. 5 and 6, the spoon exterior surface 23B is also smooth.

The mouth examination tool of the present invention will preferably be made of a single piece of injection-molded plastic using a hot molding process which automatically sterilizes the mouth examining tool for use, and preferably individually wrapped and sealed in separate sterile plastic bags right out of the mold to insure a sterile product.

In use, the present invention is an oral examination tool intended for medical and/or dental applications while performing various oral examinations of a patient. The oral examination tool of the present invention bas three distinct variations of usage in conducting oral examinations. 1. The first variation is done in a traditional and like method to a conventional Wooden tongue blade or depressant, and is accomplished by using the non-slip Textured surface 21 facing downward. This surface may be used against any area in The mouth for closer examination. The cupped convex spoon side 23B of the second end rests in the palm of a user's hand during use of the two surfaces 21 and 22 of the first end. 2. The second variation is done like the first variation, but the smooth 22 side of the present invention is utilized instead by turning the device over. This allows for smooth and less irritating surface than the textured side of the present invention for application by the user to sensitive areas in the mouth that require examination. 3. Turning the present invention around and holding it by the first end with the two-sided textured and smooth surfaces, the third variation uses the second spoon-shaped head end 23A and 23B. The spoon head when turned over and facing downward permits the user to hold down the tongue for examination of the throat by pressing the rim 24 of the spoon head against the tongue contacting the tongue outside of the gag reflex area in the center of the tongue. These cupping effects can either greatly reduce, or in some patients, even eliminate the “gag reflex” that many people suffer with. The most sensitive portion of the tongue to “gag reflex” is down the center of the tongue, and the traditional wooden and flat tongue blade or depressant goes right to this area causing a resulting gagging effect in those patients such afflicted. The dryness of a wooden tongue depressant can also add to this gagging reflex affect, whereas the non-absorbent nature of plastics being used in the tool of the present invention additionally offsets this reaction.

It is understood that the preceding description is given merely by way of illustration and not in limitation of the invention and that various modifications may be made thereto without departing from the spirit of the invention as claimed.

What is claimed is:

1. An oral examination tool having a variety of differently textured and differently shaped surfaces for contacting a variety of surfaces within a human mouth, the tool comprising:
An elongated substantially flat tool body having a first end with straight parallel sides, a width narrower than a human tongue, and a rounded end, the first end adapted for fitting within a human mouth, the first end having a smooth low friction surface on one side of the tool body adapted for contact with sensitive surface areas within a human mouth and a textured high friction surface on an opposite side of the tool body adapted for contact with slippery areas of a human mouth, and the tool body having a second end structured in the shape of a spoon head wider than the first end, the second end having a spoon rim forming curved sides extending outwardly beyond the straight parallel sides of the first end of the tool body, the spoon rim having a curved end, and the spoon rim surrounding a recessed spoon head interior surface on one side of the second end of the tool body, the spoon rim adapted for depressing a human tongue without contacting a center gag reaction part of a human tongue, and a convex spoon head exterior surface on an opposite side of the second end, the spoon head exterior adapted for fitting within a palm of a user holding the tool body, the tool body adapted for being formed of a synthetic material in a single piece by a hot molding process requiring no sterilization.

2. The tool of claim 1 wherein the textured surface comprises a pattern of protruding hemispheres.

3. The tool of claim 1 wherein the textured surface comprises a pattern of protruding pyramids.

4. The tool of claim 1 wherein the spoon head interior surface is textured.

5. The tool of claim 1 wherein the spoon head exterior surface is textured.

6. The tool of claim 1 wherein the spoon head interior surface is smooth.

7. The tool of claim 1 wherein the spoon head exterior surface is smooth.
